(19) United States
(12) Patent Application Publication
(10) Pub. No.: US 2017/0361061 A1
(43) Pub. Date: Dec. 21, 2017

(54) INFECTION RESISTANT SURFACES FOR DEVICES

(71) Applicant: Cardiac Pacemakers, Inc., St. Paul, MN (US)

(72) Inventors: Danielle Frankson, Dayton, MN (US); Gregory J. Sherwood, North Oaks, MN (US); David R. Wulfman, Minneapolis, MN (US); Kenneth Gunter, Maple Grove, MN (US); Angelo Fruci, Mahtomedi, MN (US); Adam McGraw, Mansfield, MA (US); Mary M. Byron, Roseville, MN (US)

(21) Appl. No.: 15/623,003

(22) Filed: Jun. 14, 2017

Related U.S. Application Data
(60) Provisional application No. 62/351,027, filed on Jun. 16, 2016.

(51) Int. Cl. A61M 25/00; A61L 2/02

(52) U.S. Cl. CPC A61M 25/0009; A61L 2/02; A61L 2420/02

(57) ABSTRACT

This disclosure is directed to a medical device including a surface that has been roughened to provide a roughened surface that inhibits the adhesion of microorganisms on the roughened surface.

### CROSS-REFERENCE TO RELATED APPLICATION

This application claims priority to Provisional Application No. 62/351,027, filed Jun. 16, 2016, which is herein incorporated by reference in its entirety.

### TECHNICAL FIELD

The present disclosure relates to inhibiting and preventing microorganism infections associated with devices. More specifically, the disclosure relates to infection resistant surfaces for medical devices and methods for manufacturing these surfaces.

### BACKGROUND

Some devices, including some medical devices, are implanted or inserted into humans for treating diseases or conditions. Devices such as these include catheters, pulse generators, electronic leads, and diagnostic devices. Often, the devices have one or more surfaces or surface features that provide a safe haven for microorganisms, such as bacteria. The microorganisms attach themselves to the surface of the medical device and colonize the device. When the device is inserted or implanted into the body of the patient, it introduces the microorganisms into the body. This can lead to serious and costly complications including infections, bacteremia, and endocarditis, and result in removal of any implanted device.

Medical personal and patients would welcome advances in medical devices that inhibit the adhesion and growth of microorganisms on the medical devices.

### SUMMARY

In an Example 1, a medical device including a surface that has been roughened to provide a roughened surface that inhibits the adhesion of microorganisms on the roughened surface.

In an Example 2, the medical device of Example 1, wherein the roughened surface is one of highly hydrophobic and highly hydrophilic.

In an Example 3, the medical device of any of Examples 1 and 2, wherein the roughened surface includes depressions, such that material has been removed from the surface to provide the depressions in the roughened surface.

In an Example 4, the medical device of any of Examples 1-3, wherein the roughened surface includes material that has been added to the surface to provide the roughened surface.

In an Example 5, the medical device of any of Examples 1-4, wherein the surface is at least one of a metal surface and a polymer surface.

In an Example 6, the medical device of any of Examples 1-5, wherein the roughened surface includes a layer of parylene that has been textured to provide the roughened surface.

In an Example 7, the medical device of any of Examples 1-6, wherein the roughened surface has at least one of a cross-hatch pattern, a striped pattern, and a checkerboard pattern.

In an Example 8, the medical device of any of Examples 1-7, wherein the surface has been roughened by at least one of laser patterning, chemical etching, plasma etching, physical vapor deposition coating, sputtering, and atomic layer deposition to provide the roughened surface.

In an Example 9, a method of manufacturing a medical device, the method including roughening a surface of the medical device to create a roughened surface that inhibits the adhesion of microorganisms on the roughened surface.

In an Example 10, the method of Example 9, wherein roughening the surface of the medical device includes roughening the surface to provide one of a highly hydrophobic surface and a highly hydrophilic surface that inhibits the adhesion of microorganisms on the one of the highly hydrophobic surface and the highly hydrophilic surface.

In an Example 11, the method of any of Examples 9 and 10, wherein roughening the surface of the medical device includes at least one of laser patterning, chemical etching, plasma etching, physical vapor deposition coating, sputtering, and atomic layer deposition.

In an Example 12, the method of any of Examples 9-11, wherein the surface material of the medical device includes at least one of metal and a polymer.

In an Example 13, the method of any of Examples 9-12, wherein the surface material of the medical device includes a parylene coating.

In an Example 14, the method of any of Examples 9-13, wherein roughening the surface of the medical device includes texturing a surface of a mold that is used to create the medical device, and molding the medical device in the mold to create the roughened surface.

In an Example 15, the method of any of Examples 9-13, wherein roughening the surface of the medical device comprises directly texturing material on the surface of the medical device.

In an Example 16, a medical device including a surface that has been roughened to provide a roughened surface that inhibits the adhesion of microorganisms on the roughened surface.

In an Example 17, the medical device of Example 16, wherein the roughened surface is one of highly hydrophobic and highly hydrophilic.

In an Example 18, the medical device of Example 16, wherein the roughened surface includes depressions, such that material has been removed from the surface to provide the depressions in the roughened surface.

In an Example 19, the medical device of Example 16, wherein the roughened surface includes material that has been added to the surface to provide the roughened surface.

In an Example 20, the medical device of Example 16, wherein the surface is at least one of a metal surface and a polymer surface.

In an Example 21, the medical device of Example 16, wherein the roughened surface includes a layer of parylene that has been textured to provide the roughened surface.

In an Example 22, the medical device of Example 16, wherein the roughened surface has at least one of a cross-hatch pattern, a striped pattern, and a checkerboard pattern.

In an Example 23, the medical device of Example 16, wherein the surface has been roughened by at least one of laser patterning, chemical etching, plasma etching, physical vapor deposition coating, sputtering, and atomic layer deposition to provide the roughened surface.

In an Example 24, a method of manufacturing a medical device, the method including roughening a surface of the medical device to create a roughened surface that inhibits the adhesion of microorganisms on the roughened surface.

In an Example 25, the method of Example 24, wherein roughening the surface of the medical device includes texturing a surface of a mold that is used to create the medical device, and molding the medical device in the mold to create the roughened surface.

In an Example 26, the method of Example 25, wherein the mold includes at least one of epoxy and silicone.

In an Example 27, the method of Example 24, wherein roughening the surface of the medical device comprises directly texturing a surface material of the medical device.

In an Example 28, the method of Example 27, wherein the surface material of the medical device includes at least one of metal and a polymer.

In an Example 29, the method of Example 27, wherein the surface material of the medical device includes a parylene coating that is directly textured to create the roughened surface.

In an Example 30, the method of Example 24, wherein roughening the surface of the medical device includes at least one of laser patterning, chemical etching, plasma etching, physical vapor deposition coating, sputtering, and atomic layer deposition.

In an Example 31, a method of manufacturing a medical device, the method including roughening a surface of the medical device to provide one of a highly hydrophobic surface and a highly hydrophilic surface that inhibits the adhesion of microorganisms on the one of the highly hydrophobic surface and the highly hydrophilic surface.

In an Example 32, the method of Example 31, wherein the highly hydrophobic surface provides a first water contact angle of greater than 110 degrees and the highly hydrophilic surface provides a second water contact angle of less than 10 degrees.

In an Example 33, the method of Example 31, wherein roughening the surface of the medical device includes texturing a mold surface of a mold that is used to produce the medical device, and molding the medical device in the mold.

In an Example 34, the method of Example 31, wherein roughening the surface of the medical device comprises directly texturing material on the surface of the medical device.

In an Example 35, the method of Example 31, wherein roughening the surface of the medical device includes texturing by at least one of laser etching a pattern into one of a mold and surface material of the medical device, chemical etching a pattern into one of the mold and the surface material of the medical device, plasma etching a pattern into one of the mold and the surface material of the medical device, physical vapor deposition of a pattern onto one of the mold and the surface material of the medical device, sputtering a pattern onto one of the mold and the surface material of the medical device, and atomic layer deposition of a pattern onto one of the mold and the surface material of the medical device.

While multiple embodiments are disclosed, still other embodiments of the present disclosure will become apparent to those skilled in the art from the following drawings and detailed description, which show and describe illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating a pulse generator and an electrical lead, according to embodiments of the disclosure.

FIG. 2 is a diagram illustrating an infection resistant roughened surface, according to embodiments of the disclosure.

FIG. 3 is a diagram illustrating another infection resistant roughened surface, according to embodiments of the disclosure.

FIG. 4 is a diagram illustrating another infection resistant roughened surface, according to embodiments of the disclosure.

FIG. 5 is a diagram illustrating another infection resistant roughened surface, according to embodiments of the disclosure.

FIG. 6A is a diagram illustrating a highly hydrophobic roughened surface, according to embodiments of the disclosure.

FIG. 6B is a diagram illustrating a cross-section of the highly hydrophobic roughened surface, according to embodiments of the disclosure.

FIG. 7A is a diagram illustrating a highly hydrophilic roughened surface, according to embodiments of the disclosure.

FIG. 7B is a diagram illustrating a cross-section of the highly hydrophilic roughened surface including the plateaus and the vertical troughs, according to embodiments of the disclosure.

FIG. 8A is a diagram illustrating a control coupon having a control surface that has not been patterned or textured to provide a roughened surface to inhibit the adhesion or growth of microorganisms.

FIG. 8B is a diagram illustrating colonies of bacteria cells that were grown via the procedure described above.

FIG. 9A is a diagram illustrating a textured coupon having a surface including a textured surface area, according to embodiments of the disclosure.

FIG. 9B is a diagram illustrating the resulting pattern of cell colonies on the growth plate.

FIG. 10 is a diagram illustrating a method of manufacturing a device having an infection resistant surface, according to embodiments of the disclosure.

FIG. 11 is a diagram illustrating another method of manufacturing a device having an infection resistant surface, according to embodiments of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

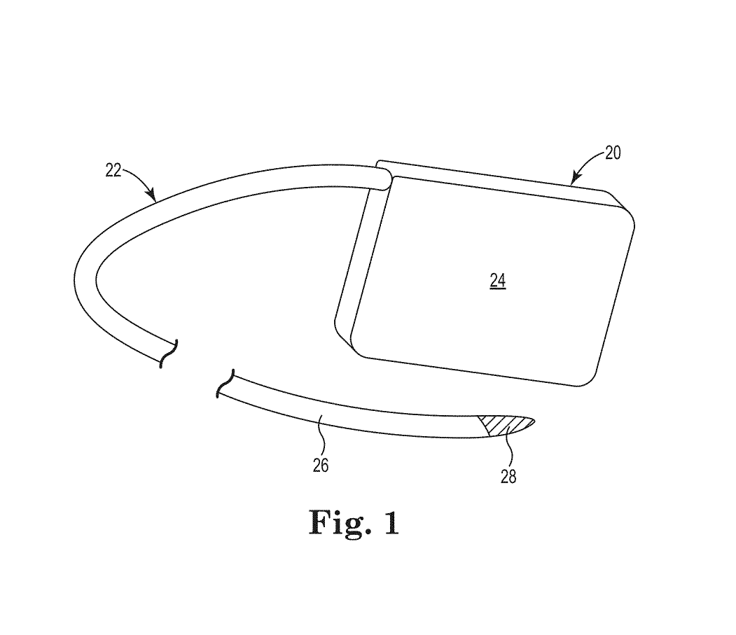

FIG. 1 is a diagram illustrating a pulse generator 20 and an electrical lead 22, according to embodiments of the disclosure. The pulse generator 20 includes at least one surface, including front surface 24, that is an infection resistant surface, and the lead 22 includes at least one surface, including lead surface 26, which is an infection resistant surface. Each of the infection resistant surfaces (of front surface 24 and lead surface 26) inhibits the adhesion and growth of microorganisms on the associated device, which reduces the chance of serious and costly complications such as infections, bacteremia, endocarditis, and removal of an implanted device.

The pulse generator 20 and the lead 22 are medical devices configured to be implanted into a human being. The pulse generator 20 can be made with a metal or a plastic/polymer, such that the front surface 24 can be a metal surface or a plastic/polymer surface. Also, the lead 22 can be made out of or with a plastic/polymer, such that the lead surface 26 is a plastic/polymer surface. In some embodiments, surfaces of the pulse generator 20 and/or the lead 22, such as the front surface 24 and the lead surface 26, include a parylene coating that acts as a biocompatible dielectric/insulator. In some embodiments, surfaces of the pulse generator 20 and/or the lead 22, such as the front surface 24 and the lead surface 26, include a polymer coating, such as a polyimide coating. In some embodiments, surfaces of the pulse generator 20 and/or the lead 22, such as the front surface 24 and the lead surface 26, include a metal substrate with a parylene coating and/or a polymer coating, such as a polyimide coating.

The pulse generator 20 and the lead 22 can be implanted for a short period of time, such as weeks or months, or for a long period of time, such as decades. The pulse generator 22 is electrically coupled to the lead 22 to provide electrical pulses at the tip 28 of the electrical lead 22 to the patient, such as to the patient's heart. In some embodiments, the pulse generator 20 is or includes a pacemaker device.

The pulse generator 20 and the lead 22 are only examples of devices that can include one or more of the infection resistant surfaces described in this disclosure. In other examples, other devices can include one or more of the infection resistant surfaces described in this disclosure, such that the infection resistant surfaces are not limited to being applied to the pulse generator 20 and/or the lead 22, or even to medical devices. Instead, the infection resistant surfaces can be put on a surface of a device that comes into contact with a human being. In some embodiments, medical devices that include one or more infection resistant surfaces include catheters, cannulas, and diagnostic devices, which may be temporarily inserted into a human and withdrawn or implanted into a patient for a short or long period of time.

Each infection resistant surface, such as front surface 24 and lead surface 26, is a roughened surface that creates an anti-favorable surface for microorganism adhesion (attachment) and growth. The roughened surface can have a random pattern or a predefined pattern with non-fouling properties. In one embodiment, an infection resistant surface is a surface of a device that has been roughened to provide a roughened surface that inhibits the adhesion of microorganisms on the roughened surface. In one embodiment, an infection resistant surface is a surface of a device that has been made one of highly hydrophobic or highly hydrophilic to inhibit the adhesion and growth of microorganisms on the device. In other embodiments, an infection resistant surface can be a highly polished surface, which is, however, difficult to maintain in a highly polished state.

The roughened surface can be created using a number of different methods. For example, an infection resistant roughened surface can be created by laser patterning, chemical etching, plasma etching, physical vapor deposition (PVD), sputter coating, atomic layer deposition (ALD), and media roughening. In some embodiments, a surface of a device is roughened by removing material, such as metal, plastic/polymer, and/or parylene, from the surface of the device. In some embodiments, the plastic/polymer removed is a polyimide. In some embodiments, a surface of a device is roughened by adding material, such as metal, plastic/polymer, and/or parylene, to the surface of the device. In some embodiments, the plastic/polymer added is a polyimide. In some embodiments, a surface of a device is roughened by texturing or patterning the surface of a mold in which the device is molded or formed, where the molded device includes the negative of the pattern created in the mold, where molding may be well suited for manufacturing lead insulation tubes, strain reliefs, suture sleeves, and epoxy headers. In some embodiments, the mold includes at least one of epoxy and silicone.

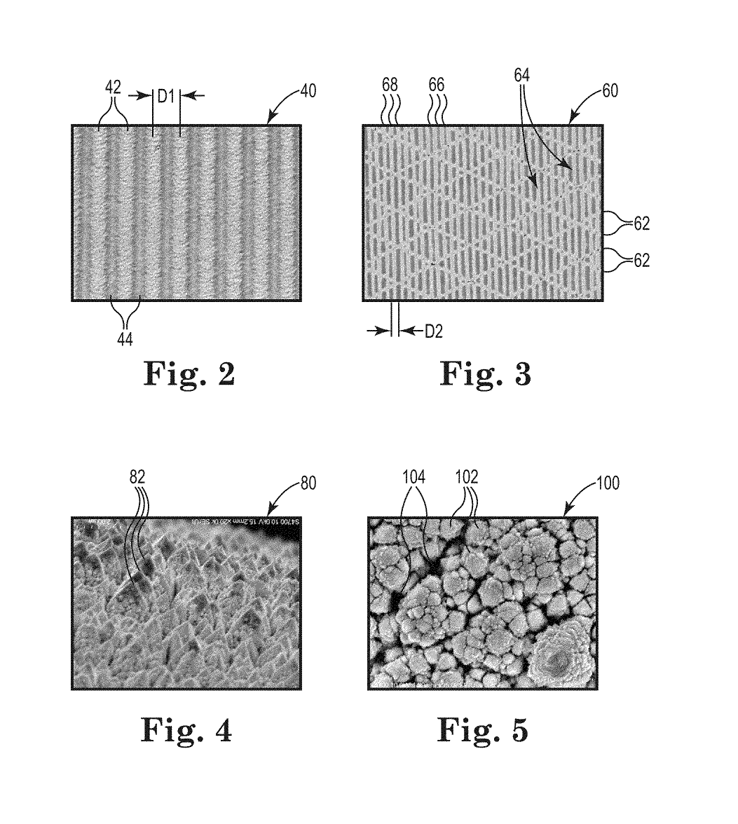

FIG. 2 is a diagram illustrating an infection resistant roughened surface 40, according to embodiments of the disclosure. The roughened surface 40 includes ribs 42 separated by troughs 44 in a vertical stripe pattern that extends from the top of FIG. 2 to the bottom of FIG. 2. The ribs 42 are separated from one another by the troughs 44 a distance D1. In some embodiments, the distance D1 is in a range of from 2 to 4 micrometers. In some embodiments, the depth of the troughs 44, from the peak or top of the ribs 42 to the bottom of the troughs 44, is in a range of from 2 to 4 micrometers. In other embodiments, the roughened surface 40 includes the ribs 42 in a stripe pattern that extends in another direction, such as horizontally from one side (the left side) to the opposing side (the right side) or diagonally across the roughened surface 40.

The roughened surface 40 is manufactured from a surface of a device, which can be or include one or more of a metal, a plastic/polymer, and a parylene covered surface. In some embodiments, the plastic/polymer is a polyimide. In one embodiment, the surface of the device is patterned and textured by laser etching the surface of the device to remove material from the surface and produce the roughened surface 40. In one embodiment, the surface of the device is patterned and textured by chemically etching the surface of the device to remove material from the surface and produce the roughened surface 40. In one embodiment, the surface of the device is patterned and textured by plasma etching the surface of the device to remove material from the surface and produce the roughened surface 40. In other embodiments, the surface of the device is patterned and textured by adding material to the surface, such as by ALD, to produce the roughened surface 40.

The roughened surface 40 can be used on devices, such as medical devices including the pulse generator 20 and the electrical lead 22. The roughened surface 40 inhibits the adhesion and growth of microorganisms on the associated device, which reduces the chance of serious and costly complications such as infections, bacteremia, endocarditis, and removal of an implanted device.

FIG. 3 is a diagram illustrating another infection resistant roughened surface 60, according to embodiments of the disclosure. The roughened surface 60 includes diagonally crisscrossed primary troughs 62 that define diamond shaped islands 64, referred to herein as a cross-hatch pattern or a diamond-hatch pattern. In some embodiments, the roughened surface 60 has a pattern that is similar to a cross-hatch pattern or diamond-hatch pattern. In some embodiments, the pattern of the roughened surface 60 is a super-hydrophilic design having a water contact angle of less than 10 degrees.

Each island 64 in the roughened surface 60 includes ribs 66 separated by secondary troughs 68 in a vertically striped pattern. The ribs 66 and secondary troughs 68 extend from the top of the island 64 to the bottom of the island 64 across the island 64, such that the ribs 66 and secondary troughs 68 increase in length, reach a maximum length, and decrease in length from one side of the island 64 to the other side of the island 64. The ribs 66 are separated from one another by the secondary troughs 68 a distance D2. In some embodiments, the distance D2 is in a range of from 2 to 8 micrometers. In some embodiments, the depth of the secondary troughs 68, from the peak or top of the ribs 66 to the bottom of the troughs 68, is in a range of from 3 to 5 micrometers. In some embodiments, the depth of the primary troughs 62, from the peak or top of the ribs 66 to the bottom of the primary troughs 62, is in a range of from 3 to 8 micrometers. In other embodiments, the roughened surface 60 includes the ribs 66 and secondary troughs 68 in a stripe pattern that extends in another direction, such as horizontally from one side (the left side) to the opposing side (the right side) or diagonally across each of the islands 64.

The roughened surface 60 is manufactured from a surface of a device, which can be or include one or more of a metal, a plastic/polymer, and a parylene covered surface. In some embodiments, the plastic/polymer is a polyimide. In one embodiment, the surface of the device is patterned and textured by laser etching the surface of the device to remove material from the surface and produce the roughened surface 60. In one embodiment, the surface of the device is patterned and textured by chemically etching the surface of the device to remove material from the surface and produce the roughened surface 60. In one embodiment, the surface of the device is patterned and textured by plasma etching the surface of the device to remove material from the surface and produce the roughened surface 60. In other embodiments, the surface of the device is patterned and textured by adding material to the surface, such as by ALD, to produce the roughened surface 60.

The roughened surface 60 can be used on devices, such as medical devices including the pulse generator 20 and the electrical lead 22. The roughened surface 60 inhibits the adhesion and growth of microorganisms on the associated device, which reduces the chance of serious and costly complications such as infections, bacteremia, endocarditis, and removal of an implanted device.

FIG. 4 is a diagram illustrating another infection resistant roughened surface 80, according to embodiments of the disclosure. The roughened surface 80 includes bumps or nodules 82 in a random pattern across the roughened surface 80. The nodules 82 have peaks and define valleys situated between the nodules 82. In some embodiments, the depth of the valleys, from the top of the peaks to the troughs of the valleys, is in a range of from 2 to 4 micrometers.

The roughened surface 80 is manufactured from a surface of a device, which can be or include one or more of a metal, a plastic/polymer, and a parylene covered surface. In some embodiments, the plastic/polymer is a polyimide. In one embodiment, the surface of the device is textured by adding material to the surface, such as by PVD and/or sputter coating, to produce the roughened surface 80. In other embodiments, the surface of the device can be pseudo-randomly patterned and textured by removing material from the surface, such as by laser etching, chemical etching, and plasma etching, to produce the roughened surface 80. In other embodiments, the surface of the device can be pseudo-randomly patterned and textured by adding material to the surface, such as by ALD, to produce the roughened surface 80.

The roughened surface 80 can be used on devices, such as medical devices including the pulse generator 20 and the electrical lead 22. The roughened surface 80 inhibits the adhesion and growth of microorganisms on the associated device, which reduces the chance of serious and costly complications such as infections, bacteremia, endocarditis, and removal of an implanted device.

FIG. 5 is a diagram illustrating another infection resistant roughened surface 100, according to embodiments of the disclosure. This roughened surface 100 includes nodules 102 in a random pattern across the roughened surface 100. The nodules 102 define random valleys 104 between nodules 102 and groups of nodules 102. In some embodiments, the depth of the random valleys 104, from the top of the nodules 102 to the troughs of the valleys 104, is in a range of from 2 to 4 micrometers.

The roughened surface 100 is manufactured from a surface of a device, which can be or include one or more of a metal, a plastic/polymer, and a parylene covered surface. In some embodiments, the plastic/polymer is a polyimide. In one embodiment, the surface of the device is textured by adding material to the surface, such as by PVD and/or sputter coating, to produce the roughened surface 100. In other embodiments, the surface of the device can be pseudo-randomly patterned and textured by removing material from the surface, such as by laser etching, chemical etching, and plasma etching, to produce the roughened surface 100. In other embodiments, the surface of the device can be pseudo-randomly patterned and textured by adding material to the surface, such as by ALD, to produce the roughened surface 100.

The roughened surface 100 can be used on devices, such as medical devices including the pulse generator 20 and the electrical lead 22. The roughened surface 100 inhibits the adhesion and growth of microorganisms on the associated device, which reduces the chance of serious and costly complications such as infections, bacteremia, endocarditis, and removal of an implanted device.

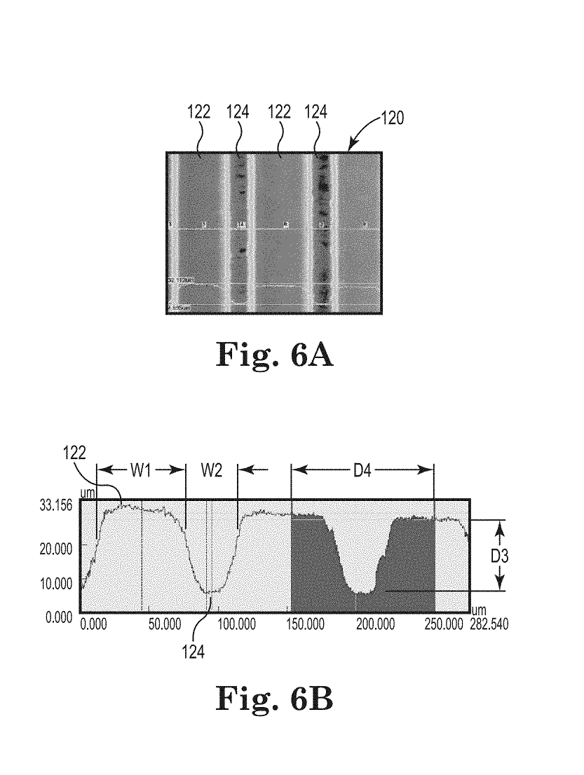

FIG. 6A is a diagram illustrating a highly hydrophobic roughened surface 120, according to embodiments of the disclosure. The highly hydrophobic roughened surface 120 includes ribs 122 separated by troughs 124 in a vertical stripe pattern that extends from the top of FIG. 6A to the bottom of FIG. 6A. In other embodiments, the roughened surface 120 can include the ribs 122 and troughs 124 in a stripe pattern that extends in another direction, such as horizontally from one side, such as the left side, to the opposing side, the right side, or diagonally across the roughened surface 120.

FIG. 6B is a diagram illustrating a cross-section of the highly hydrophobic roughened surface 120, according to embodiments of the disclosure. Each of the ribs 122 has a width W1 and each of the troughs 124 has a width W2. Also, each of the troughs has a depth D3, as measured from the peak or top of the ribs 122 to the bottom of the troughs 124. The ribs 122 are separated from one another by the troughs 124 a distance D4, as taken from the midline of adjacent ribs 122. The highly hydrophobic roughened surface 120 has a water contact angle in a range of from 110 to 150 degrees. In some embodiments, the width W1 of each of the ribs 122 is in a range of from 25 to 75 micrometers. In some embodiments, the width W2 of each of the troughs 124 is in a range of from 25 to 75 micrometers. In some embodiments, the depth D3 of each of the troughs 124 is in a range of from 10 to 30 micrometers. In some embodiments, the distance D4 between adjacent ribs 122 is in a range of from 75 to 125 micrometers.

The roughened surface 120 is manufactured from a surface of a device, which can be or include one or more of a metal, a plastic/polymer, and a parylene covered surface. In some embodiments, the plastic/polymer is a polyimide. In one embodiment, the surface of the device is patterned and textured by laser etching the surface of the device to remove material from the surface and produce the roughened surface 120. In one embodiment, the surface of the device is patterned and textured by chemically etching the surface of the device to remove material from the surface and produce the roughened surface 120. In one embodiment, the surface of the device is patterned and textured by plasma etching the surface of the device to remove material from the surface and produce the roughened surface 120. In other embodiments, the surface of the device is patterned and textured by adding material to the surface, such as by ALD, to produce the roughened surface 120.

The highly hydrophobic roughened surface 120 can be used on devices, such as medical devices including the pulse generator 20 and the electrical lead 22. The roughened surface 120 inhibits the adhesion and growth of microorganisms on the associated device, which reduces the chance of serious and costly complications such as infections, bacteremia, endocarditis, and removal of an implanted device.

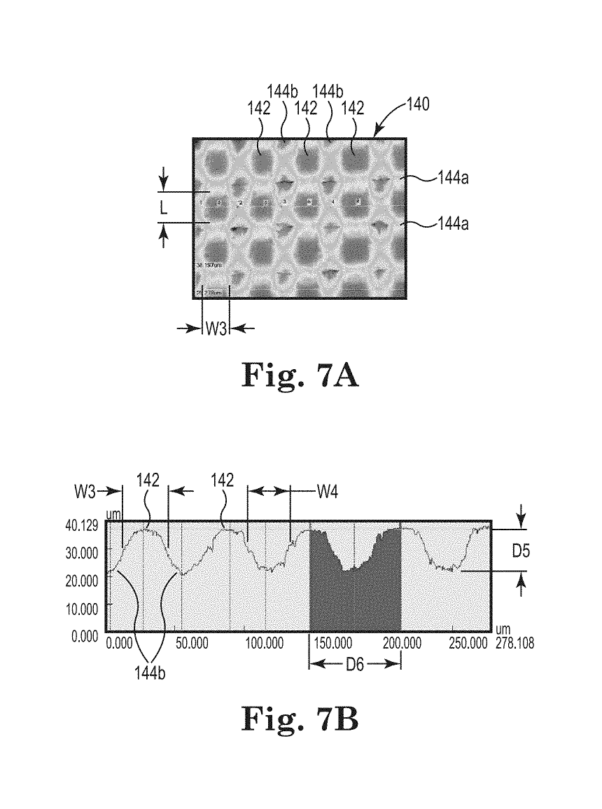

FIG. 7A is a diagram illustrating a highly hydrophilic roughened surface 140, according to embodiments of the disclosure. The highly hydrophilic roughened surface 140 includes plateaus 142 separated by horizontal troughs 144a and vertical troughs 144b, to produce a checkerboard pattern.

FIG. 7B is a diagram illustrating a cross-section of the highly hydrophilic roughened surface 140 including the plateaus 142 and the vertical troughs 144b, according to embodiments of the disclosure. Each of the plateaus 142 has a length L (as shown in FIG. 7A) and a width W3, and each of the troughs 144b has a width W4. Also, each of the troughs 144b has a depth D5, as measured from the peak or top of the plateaus 142 to the bottom of the troughs 144b. The plateaus 142 are separated from one another by the troughs 144b a distance D6, as taken from the middle of adjacent plateaus 142. The highly hydrophilic roughened surface 140 has a water contact angle in a range of from 0 to 10 degrees. In some embodiments, the length L of each of the plateaus 142 is in a range of from 20 to 60 micrometers and the width W3 of each of the plateaus 142 is in a range of from 20 to 60 micrometers. In some embodiments, the width W4 of each of the troughs 144b is in a range of from 20 to 60 micrometers. In some embodiments, the depth D5 of each of the troughs 144b is in a range of from 10 to 30 micrometers. In some embodiments, the distance D6 between adjacent plateaus 142 is in a range of from 40 to 80 micrometers.

The roughened surface 140 is manufactured from a surface of a device, which can be or include one or more of a metal, a plastic/polymer, and a parylene covered surface. In some embodiments, the plastic/polymer is a polyimide. In one embodiment, the surface of the device is patterned and textured by laser etching the surface of the device to remove material from the surface and produce the roughened surface 140. In one embodiment, the surface of the device is patterned and textured by chemically etching the surface of the device to remove material from the surface and produce the roughened surface 140. In one embodiment, the surface of the device is patterned and textured by plasma etching the surface of the device to remove material from the surface and produce the roughened surface 140. In other embodiments, the surface of the device is patterned and textured by adding material to the surface, such as by ALD, to produce the roughened surface 140.

The highly hydrophilic roughened surface 140 can be used on devices, such as medical devices including the pulse generator 20 and the electrical lead 22. The roughened surface 140 inhibits the adhesion and growth of microorganisms on the associated device, which reduces the chance of serious and costly complications such as infections, bacteremia, endocarditis, and removal of an implanted device.

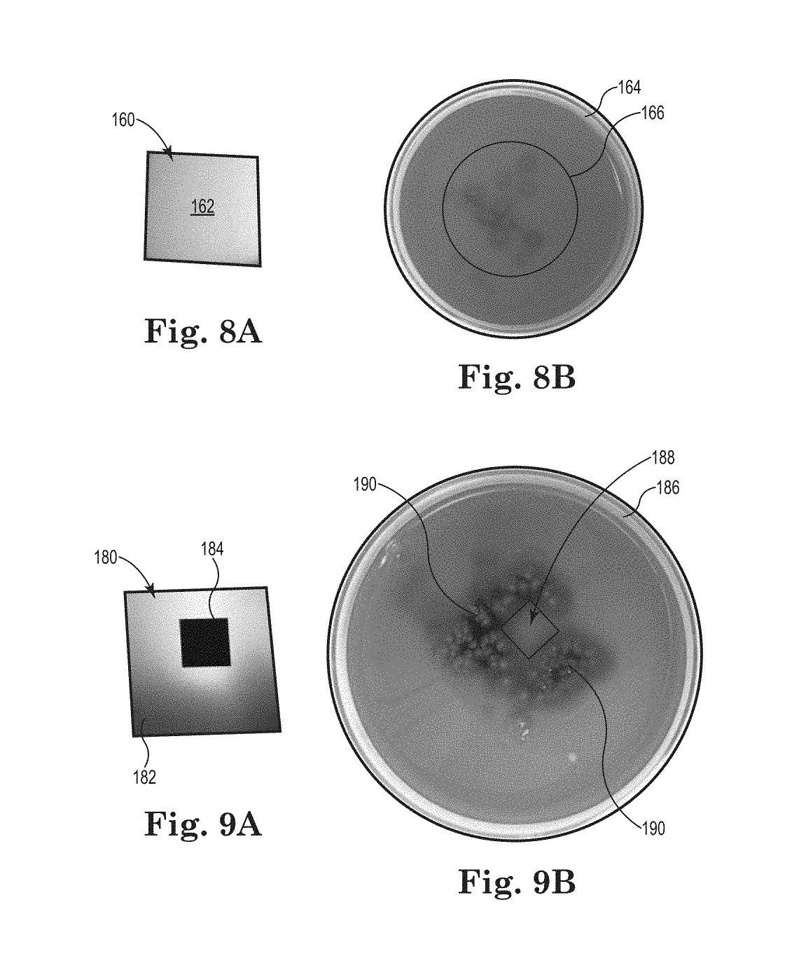

FIG. 8A is a diagram illustrating a control coupon 160 having a control surface 162 that has not been patterned or textured to provide a roughened surface to inhibit the adhesion or growth of microorganisms. The control coupon 160 was used in testing to provide a non-textured control sample. In some embodiments, the control coupon 160 includes or is made out of titanium.

In embodiments, the control coupon 160 was incubated for a period of time, such as 2 or more hours, with liquid cultures of bacteria, such as E. Coli and/or S. Aureus. After the incubation period, the control coupon 160 was washed to remove loosely attached cells and then stamped face down onto a growth plate 164. The cells transferred to the growth plate 164 were grown out for a growth time and at a growth temperature, such as overnight at 37 degrees Celsius or for 72 hours at 25 degrees Celsius, to provide quantifiable colonies of cells.

FIG. 8B is a diagram illustrating colonies of bacteria cells 166 that were grown via the procedure described above. As illustrated, the control coupon 160 with the non-textured control surface 162 produced or resulted in a number of bacteria colonies 166.

FIG. 9A is a diagram illustrating a textured coupon 180 having a surface 182 including a textured surface area 184, according to embodiments of the disclosure. The textured surface area 184 has been textured to provide a roughened surface that inhibits the adhesion and growth of microorganisms. The textured surface area 184 is a substantially square or rectangular region including a roughened surface as described in this disclosure. In some embodiments, the textured coupon 180 includes or is made out of titanium.

In embodiments, the textured coupon 180 was incubated and cells were grown as described above for the control coupon 160. The textured coupon 180 was incubated for a period of time, such as 2 or more hours, with liquid cultures of bacteria, such as E. Coli and/or S. Aureus. After the incubation period, the textured coupon 180 was washed to remove loosely attached cells and then stamped face down onto a growth plate 186. The cells transferred to the growth plate 186 were grown out for a growth time and at a growth temperature, such as overnight at 37 degrees Celsius or for 72 hours at 25 degrees Celsius, to provide quantifiable colonies of cells.

FIG. 9B is a diagram illustrating the resulting pattern of cell colonies on the growth plate 186. As illustrated, few or no cell colonies were grown in an area 188 corresponding to the textured surface area 184 of the textured coupon 180. However, the surrounding area 190, which corresponds to non-textured portions of the textured coupon 180, includes a number of cell colonies produced as a result of the above process.

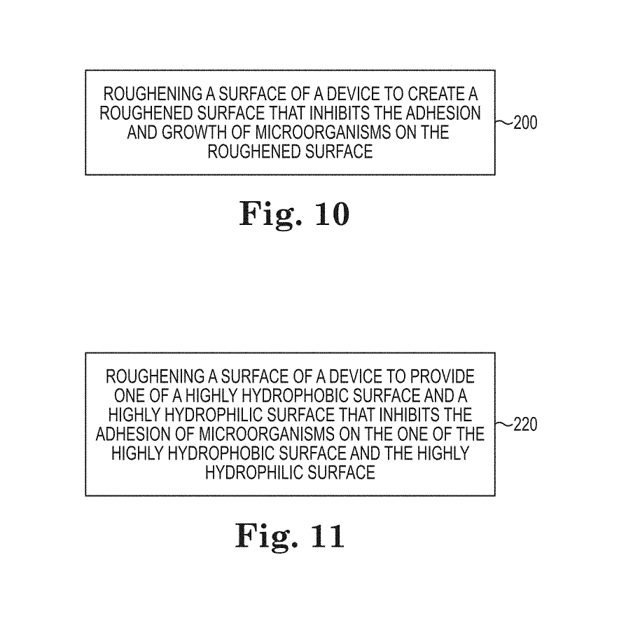

FIG. 10 is a diagram illustrating a method of manufacturing a device having an infection resistant surface, according to embodiments of the disclosure. The device has a surface that is made into the infection resistant surface. In some embodiments, the device is a medical device, such as the pulse generator 20 or the electrical lead 22 (shown in FIG. 1). In some embodiments, the surface of the device is similar to the front surface 24 of the pulse generator 20 and/or the lead surface of the electrical lead 22.

The method, at 200, includes the step of roughening the surface of the device to create a roughened surface that inhibits the adhesion and growth of microorganisms on the roughened surface. Where, inhibiting the adhesion and growth of microorganisms on the roughened surface reduces the chance of serious and costly complications such as infections, bacteremia, endocarditis, and removal of an implanted device.

The surface can be roughened using one or more of the methods or procedures described above.

In some embodiments, roughening the surface of the device includes directly texturing the surface material of the device. In some embodiments, the surface material of the device includes at least one of metal and a polymer. In some embodiments, the polymer is a polyimide. In some embodiments, the surface material of the device includes a parylene coating that is directly textured to create the roughened surface.

In some embodiments, roughening the surface of the device includes texturing a surface of a mold that is used to create the device and molding the device in the mold to create the roughened surface. In some embodiments, the mold includes at least one of epoxy and silicone.

In some embodiments, roughening the surface of the device includes at least one of laser etching a pattern into a mold or the surface material of the device, chemically etching a pattern into one of the mold or the surface material of the device, plasma etching a pattern into one of the mold or the surface material of the device, PVD coating a pattern onto one of the mold or the surface material of the device; sputtering or sputter coating a pattern onto one of the mold or the surface material of the device, and ALD of a pattern onto one of the mold or the surface material of the device.

FIG. 11 is a diagram illustrating another method of manufacturing a device having an infection resistant surface, according to embodiments of the disclosure. The device has a surface that is made into the infection resistant surface. In some embodiments, the device is a medical device, such as the pulse generator 20 or the electrical lead 22 (shown in FIG. 1). In some embodiments, the surface of the device is similar to the front surface 24 of the pulse generator 20 and/or the lead surface of the electrical lead 22.

The method, at 220, includes the step of roughening a surface of the device to provide one of a highly hydrophobic surface and a highly hydrophilic surface that inhibits the adhesion of microorganisms on the one of the highly hydrophobic surface and the highly hydrophilic surface. Where, inhibiting the adhesion and growth of microorganisms reduces the chance of serious and costly complications such as infections, bacteremia, endocarditis, and removal of an implanted device. In some embodiments, the highly hydrophobic surface provides a water contact angle in a range of from 110 to 150 degrees. In some embodiments, the highly hydrophilic surface provides a water contact angle in a range of from 0 to 10 degrees. In some embodiments, the highly hydrophobic surface provides a water contact angle of greater than 110 degrees. In some embodiments, the highly hydrophilic surface provides a water contact angle of less than 10 degrees.

As described above for the previous method, the surface can be roughened using one or more of the methods or procedures described above.

In some embodiments, roughening the surface of the device includes directly texturing the surface material of the device. In some embodiments, the surface material of the device includes at least one of metal and a polymer. In some embodiments, the polymer is a polyimide. In some embodiments, the surface material of the device includes a parylene coating that is directly textured to create the roughened surface.

In some embodiments, roughening the surface of the device includes texturing a surface of a mold that is used to create the device and molding the device in the mold to create the roughened surface. In some embodiments, the mold includes at least one of epoxy and silicone.

In some embodiments, roughening the surface of the device includes at least one of laser etching a pattern into a mold or the surface material of the device, chemically etching a pattern into one of the mold or the surface material of the device, plasma etching a pattern into one of the mold or the surface material of the device, PVD coating of a pattern onto one of the mold or the surface material of the device; sputtering or sputter coating a pattern onto one of the mold or the surface material of the device, and ALD of a pattern onto one of the mold or the surface material of the device.

Various modifications and additions can be made to the exemplary embodiments discussed without departing from the scope of the disclosure. For example, while the embodiments described above refer to particular features, the scope of this disclosure also includes embodiments having different combinations of features and embodiments that do not include all of the described features. Accordingly, the scope of the disclosure is intended to embrace all such alternatives, modifications, and variations as fall within the scope of the claims, together with all equivalents thereof.

We claim:

1. A medical device, comprising:
a surface that has been roughened to provide a roughened surface that inhibits the adhesion of microorganisms on the roughened surface.

2. The medical device of claim 1, wherein the roughened surface is one of highly hydrophobic and highly hydrophilic.

3. The medical device of claim 1, wherein the roughened surface includes depressions, such that material has been removed from the surface to provide the depressions in the roughened surface.

4. The medical device of claim 1, wherein the roughened surface includes material that has been added to the surface to provide the roughened surface.

5. The medical device of claim 1, wherein the surface is at least one of a metal surface and a polymer surface.

6. The medical device of claim 1, wherein the roughened surface includes a layer of parylene that has been textured to provide the roughened surface.

7. The medical device of claim 1, wherein the roughened surface has at least one of a cross-hatch pattern, a striped pattern, and a checkerboard pattern.

8. The medical device of claim 1, wherein the surface has been roughened by at least one of laser patterning, chemical etching, plasma etching, physical vapor deposition coating, sputtering, and atomic layer deposition to provide the roughened surface.

9. A method of manufacturing a medical device, the method comprising:
roughening a surface of the medical device to create a roughened surface that inhibits the adhesion of microorganisms on the roughened surface.

10. The method of claim 9, wherein roughening the surface of the medical device comprises:
texturing a surface of a mold that is used to create the medical device; and
molding the medical device in the mold to create the roughened surface.

11. The method of claim 10, wherein the mold includes at least one of epoxy and silicone.

12. The method of claim 9, wherein roughening the surface of the medical device comprises directly texturing a surface material of the medical device.

13. The method of claim 12, wherein the surface material of the medical device includes at least one of metal and a polymer.

14. The method of claim 12, wherein the surface material of the medical device includes a parylene coating that is directly textured to create the roughened surface.

15. The method of claim 9, wherein roughening the surface of the medical device comprises at least one of: laser patterning; chemical etching; plasma etching; physical vapor deposition coating; sputtering; and atomic layer deposition.

16. A method of manufacturing a medical device, the method comprising:
roughening a surface of the medical device to provide one of a highly hydrophobic surface and a highly hydrophilic surface that inhibits the adhesion of microorganisms on the one of the highly hydrophobic surface and the highly hydrophilic surface.

17. The method of claim 16, wherein the highly hydrophobic surface provides a first water contact angle of greater than 110 degrees and the highly hydrophilic surface provides a second water contact angle of less than 10 degrees.

18. The method of claim 16, wherein roughening the surface of the medical device comprises:
texturing a mold surface of a mold that is used to produce the medical device; and
molding the medical device in the mold.

19. The method of claim 16, wherein roughening the surface of the medical device comprises directly texturing material on the surface of the medical device.

20. The method of claim 16, wherein roughening the surface of the medical device comprises texturing by at least one of: laser etching a pattern into one of a mold and surface material of the medical device: chemical etching a pattern into one of the mold and the surface material of the medical device; plasma etching a pattern into one of the mold and the surface material of the medical device; physical vapor deposition of a pattern onto one of the mold and the surface material of the medical device; sputtering a pattern onto one of the mold and the surface material of the medical device; and atomic layer deposition of a pattern onto one of the mold and the surface material of the medical device.
